(19) United States
(12) Patent Application Publication
(10) Pub. No.: US 2024/0397027 A1
(43) Pub. Date: Nov. 28, 2024

(54) RELEASABLY COUPLABLE DIGITAL STEREO IMAGE DISPLAY DEVICE, SYSTEM HAVING SAME, AND DIGITAL SURGICAL MICROSCOPE

(71) Applicant: Carl Zeiss Meditec AG, Jena (DE)

(72) Inventors: Christian Wolf, Aalen (DE); Hubert Katzer, Koenigsbronn-Ochsenberg (DE); Michael Wirth, Aalen (DE); Daniel Kolster, Oberkochen (DE)

(21) Appl. No.: 18/674,410

(22) Filed: May 24, 2024

(30) Foreign Application Priority Data
May 26, 2023 (DE) 10 2023 113 897.9

(51) Int. Cl. H04N 13/30; A61B 90/00; G02B 21/22

(52) U.S. Cl. CPC H04N 13/30; A61B 90/37; G02B 21/22; A61B 2090/372; H04N 2213/001

(57) ABSTRACT

A digital stereo image display device has a holding arm which includes a proximal end for attachment to a mount for an optical system with a digital image recording device and which includes a distal end where two digital eyepieces are present and configured to display the images captured by the image recording device from two different viewing angles. An interface part of a quick release interface for releasable attachment to the mount is present at the proximal end. A system includes the digital image display device and a mount. A digital surgical microscope includes the system and a digital image recording device.

### CROSS-REFERENCE TO RELATED APPLICATIONS

This application claims priority of German patent application no. 10 2023 113 897.9, filed May 26, 2023, the entire content of which is incorporated herein by reference.

### TECHNICAL FIELD

The disclosure relates to a digital stereo image display device having a holding arm which includes a proximal end for attachment to a mount for an optical system with a digital image recording device and which includes a distal end where two digital eyepieces are present and configured to display the images captured by the image recording device from two different viewing angles. The disclosure also relates to a system made of such a digital stereo image display device and a mount. Further, the disclosure relates to a digital surgical microscope having a corresponding system.

### BACKGROUND

In microsurgical procedures, especially within the scope of ophthalmology, stereo microscopes with conventional eyepieces are increasingly being replaced by digital image recording devices and digital image display devices for displaying the stereoscopic images captured by the image recording device from two different viewing directions. In this context, two video cameras are used as image recording device, with the captured images then being displayed on a stereoscopic electronic visual display, that is, an electronic visual display capable of displaying three-dimensional images. Such digital microscopes are also referred to as digiscopes and create ergonomically better conditions for the surgeon.

A pair of display glasses is frequently also used in place of a stereoscopic electronic visual display, for instance in a head-mounted visualization system as described in US 2023/0087402 and US 2023/0088437 by the applicant.

In ophthalmology, cataract operations are performed with a tight schedule, with the result that the surgeon wears the visualization system (head-mounted display) on their head for several hours a day. For surgeons, this is quite a load in terms of weight over the course of a day. Thus, the use of a stereoscopic electronic visual display would be advantageous for these surgeons. However, not everybody is able to recognize three-dimensional images on the stereoscopic electronic visual display, and so these people require an alternative.

### SUMMARY

It is an object of the present disclosure to remove the disadvantages described in the prior art.

This object is, for example, achieved by a digital stereo image display device having: a holding arm having a proximal end for attachment to a mount for an optical system with a digital image recording device; the holding arm further having a distal end where two digital eyepieces are disposed and configured to display images captured by the digital image recording device from two different viewing angles; and, an interface part of a quick release interface for releasable attachment to the mount being present at the proximal end.

The above-mentioned object is, for example, further achieved by a system including: a mount; a digital stereo image display device having a holding arm having a proximal end for attachment to the mount for an optical system with a digital image recording device; the holding arm further having a distal end where two digital eyepieces are disposed and configured to display images captured by the digital image recording device from two different viewing angles; the digital stereo image display device further having a first interface part of a quick release interface for releasable attachment to the mount being disposed at the proximal end; and, the mount having a second interface part of the quick release interface, wherein the second interface part is configured to mate with the first interface part of the digital stereo image display device.

The above-mentioned object is, for example, further achieved by a digital surgical microscope including: a digital image recording device; a system including a mount and a digital stereo image display device; the digital stereo image display device having a holding arm having a proximal end for attachment to the mount for an optical system with the digital image recording device; the holding arm further having a distal end where two digital eyepieces are disposed and configured to display images captured by the digital image recording device from two different viewing angles; the digital stereo image display device further having a first interface part of a quick release interface for releasable attachment to the mount being disposed at the proximal end; and, the mount having a second interface part of the quick release interface, wherein the second interface part is configured to mate with the first interface part of the digital stereo image display device.

At its proximal end of the holding arm, the digital stereo image display device according to the disclosure includes an interface part of a quick release interface for releasable attachment to the mount.

In surgical situations in which greater immersion is desired, the surgeon is thus able to resort to the image of the stereo image display device such that they can “mask” their surroundings. The use of the releasably attachable or releasably couplable digital stereo image display device is also advantageous for surgeons who have difficulties with recognizing a three-dimensional image on the stereoscopic electronic visual display.

A quick release interface should be understood to mean, in particular, an interface that can be connected and separated again without much outlay, and preferably without tools.

So as to be able to provide particularly ergonomic posture for the surgeon, at least one joint is present between the proximal end and the distal end of the holding arm. This joint facilitates the operation.

For ergonomic use of the stereo image display device in ophthalmology, it is advantageous for the digital eyepieces to each be positively guided in a plane.

It is advantageous for the joint to include a joint axis such that a distal partial arm adjoining the distal end is able to be pivoted in a common plane vis-à-vis a proximal partial arm adjoining the proximal end. Thus, this reduces the number of degrees of freedom allowing a fast, simple, and ergonomic positioning of the digital stereo image display device. For reasons of space, it may be preferable in this context for the holding arm to be attached to a back side of the housing for the digital eyepieces. Then, attachment is thus present in the middle or centrally on the digital eyepieces.

Alternatively, it is possible that the joint includes a joint axis such that a distal partial arm adjoining the distal end is able to be pivoted in a first plane which is arranged parallel to a second plane in which a proximal partial arm adjoining the proximal end is able to be pivoted. This allows realization of an articulated chain extending between the proximal end and the distal end. For reasons of space, it may be preferable in this context for the holding arm to be attached to a side of the housing for the digital eyepieces. Then, attachment is thus present off-center on the digital eyepieces, whereby the articulated chain can also be referred to as a “lateral” articulated chain.

An ergonomic work position for a surgeon can further be realized if a respective further joint is present at the proximal partial arm and/or at the distal partial arm, the respective further joint allowing further pivoting of the digital eyepieces in the plane. This joint is preferably a rotary joint.

Further, there is also the option of the holding arm being of a multi-part and telescopic form. For instance, in this case a realization of the holding arm with two joints is considered, with the result that the holding arm can only be pivoted in one plane. Moreover, the holding arm may be of telescopic form, for example by using a carriage. In this case, a corresponding scissor mechanism purposefully restricts the degrees of freedom relating to the mobility of the stereo image display device. This facilitates positioning.

To be able to fix the pose of the digital eyepieces in space, it is advantageous if the joint and/or else all further joints are assigned a releasable locking means. This allows the joints to be changed between a mobile configuration and an immobile configuration, as required. If the mobile configuration is adopted, the digital eyepieces can easily be repositioned. If the immobile configuration is adopted as a result of locking the locking means, the current pose of the digital eyepieces in space is set. An intermediate stage is also possible, in which the digital eyepieces can be pivoted against a frictional force, with their own weight however being insufficient for an adjustment; that is, an independent adjustment is prevented.

While head-mounted displays (HMDs) always require a repository surface when not worn by the surgeon, the present stereo image display device is connected to the advantage that the latter can simply be folded upwards. It requires no repository surface.

To be able to quickly attach the stereo image display device to a mount, it was found to be advantageous if the quick release interface forms an interlocking connection in the coupled state. By preference, a quick release interface in the form of a dovetail joint with a releasable latching device is considered in this case. For instance, a so-called V-lock might be considered here, the latter moreover facilitating the introducibility of the interface part of the stereo image display device as a result of its v-shaped configuration of the dovetail. In this case, the latching device includes a latching member which latches in a corresponding latching cutout when the quick release interface is connected. In this case, the latching cutout can be assigned to one of mount and holding arm, with the latching member then being assigned to the other of mount and holding arm.

By preference, the interface part includes electrical contacts and moreover is configured to establish a data connection with the image recording device and/or with a control computer coupled to the image recording device when the quick release interface is closed. Consequently, mechanical and electrical coupling of the stereo image display device to a mount or to a surgical microscope is thus enabled.

To preclude loose cables, as are typically present in a head-mounted display, the holding arm may in this case provide a channel for guiding electrical lines. To this end, the holding arm can be formed to have a u-shaped cross section. Then, the cables are guided in the interior “u”. Thus, a u-shaped cable channel is present in the latter case. The cable channel may also have any other desired cross-sectional form, for instance a square cross-sectional form.

The configurations, advantages and effects explained in the context of the stereo image display device according to the disclosure equally apply to a system according to the disclosure made of such a digital stereo image display device and a mount, which in turn includes an interface part of the quick release interface which mates with the interface part of the stereo image display device.

This thus allows the digital stereo image display device to be assembled on the mount very quickly or else be easily released from the mount again. This procedure is also of interest if, for instance, an assistant would also need to look at the operating site.

It is advantageous if the mating interface part of the mount is assigned a securing device for additionally securing the coupled interface. This thus prevents the stereo image display device from undesirably or inadvertently detaching from the mount. For instance, a securing screw for blocking an actuation of the latching device is considered in this case. In this case, the mating interface part of the mount may be formed at different locations on the mount and, in particular, also be provided multiple times. This then also allows different and/or multiple stereo image display devices to be attached to the mount. If the mating interface parts of the mount are provided multiple times and, for instance, formed on different side faces of the mount, then the stereo image display device can be attached to the mount in different orientations.

The configurations, advantages and effects explained in the context of the stereo image display device according to the disclosure and in the context of the system according to the disclosure equally apply to the digital surgical microscope according to the disclosure. The latter is provided with such a system and with a digital image recording device and with an image display device. In this case, the digital image recording device is preferably a digiscope, wherein a stereoscopic electronic visual display may preferably be present as image display device.

### BRIEF DESCRIPTION OF DRAWINGS

The invention will now be described with reference to the drawings wherein:

FIG. 1 shows a perspective view of a digital surgical microscope;

FIG. 2 shows a perspective of the detail A from FIG. 1, to which a first variant of a digital stereo image display device is attached in a first position (parked position);

FIG. 3 shows an illustration corresponding to FIG. 2 of the stereo image display device in a second position (work position);

FIG. 4 shows an illustration corresponding to FIG. 3 of a second variant of a digital stereo image display device in the second position;

FIG. 5 shows a detailed view of the quick release interface from a first direction;

FIG. 6 shows the detailed view of the quick release interface from FIG. 5 from a second direction; and,

FIG. 7 shows a further variant of a digital surgical microscope in which a lateral articulated chain is present, the adjacent chain members of which are pivotable relative to one another in parallel planes.

### DETAILED DESCRIPTION

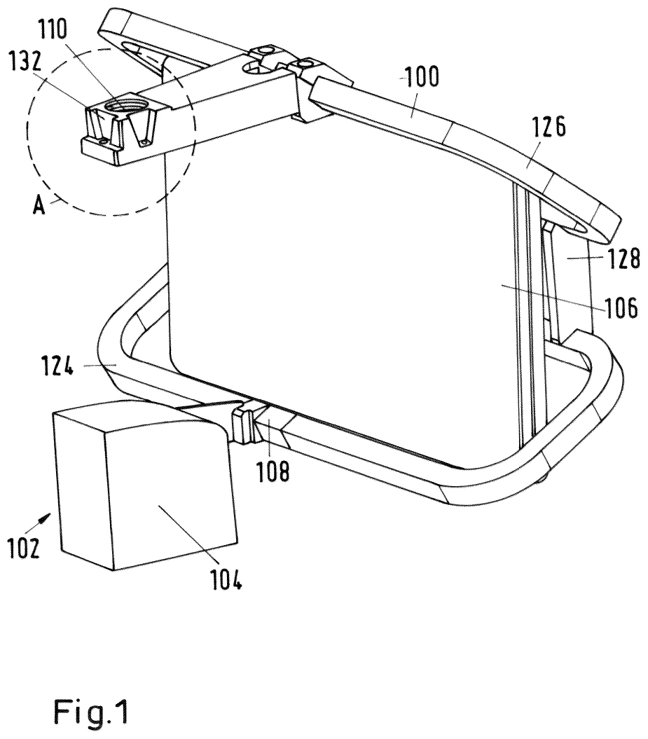

FIG. 1 shows an optical system 102 which is attached to a mount 100. The optical system 102 includes a digital image recording device 104, which is only depicted schematically. This image recording device 104 is configured to capture images of the operating site from two different viewing angles and accordingly transfer these to a control computer. In the present case, the captured images are for instance transferred to an image display device 106, in particular to a stereoscopic electronic visual display.

Purely by way of example, the mount 100 itself includes a first bracket 124 and a second bracket 126, which are rigidly connected to one another via a plurality of connection pieces 128. The mount 100 includes a first attachment device 108 for attaching the image recording device 104 and a second attachment device 110 for attaching the mount 100 to the ceiling, to a stand, or to a frame. Moreover, a third attachment device not shown in any more detail is present and serves for attaching the image display device 106.

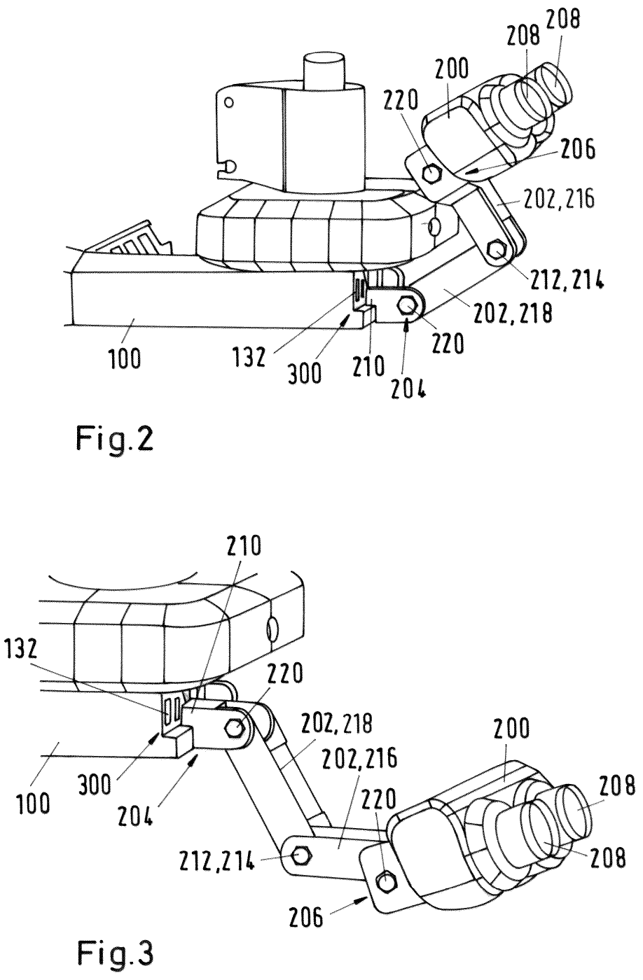

The mount 100 includes a first interface part 132 of a quick release interface 300 for attaching a digital stereo image display device 200, as shown in detail in FIGS. 2 to 6. In this case, the first interface part 132 can be present on the mount 100 on an end face, wherein a lateral arrangement-oriented perpendicular to the end face-of the first interface part 132 is also possible, as likewise illustrated by the figures. The opposite side-oriented perpendicular to the end face-of the mount 100 not visible here may also include such a first interface part 132.

The digital stereo image display device 200 can be identified in FIG. 2, it includes a holding arm 202 which has a proximal end 204 for attachment to the mount 100 and a distal end 206, wherein two digital eyepieces 208 are present at the distal end 206 and configured to display the images captured by the image recording device 104 from two different viewing angles. For the sake of clarity, the first interface part 132 on the mount 100 is only shown in part. At the proximal end 204, the digital stereo image display device 200 likewise includes a second interface part 210 of the quick release interface 300, which is formed so as to mate with the first interface part 132 on the mount 100 such that the digital stereo image display device 200 is releasably attachable or attached to the mount 100.

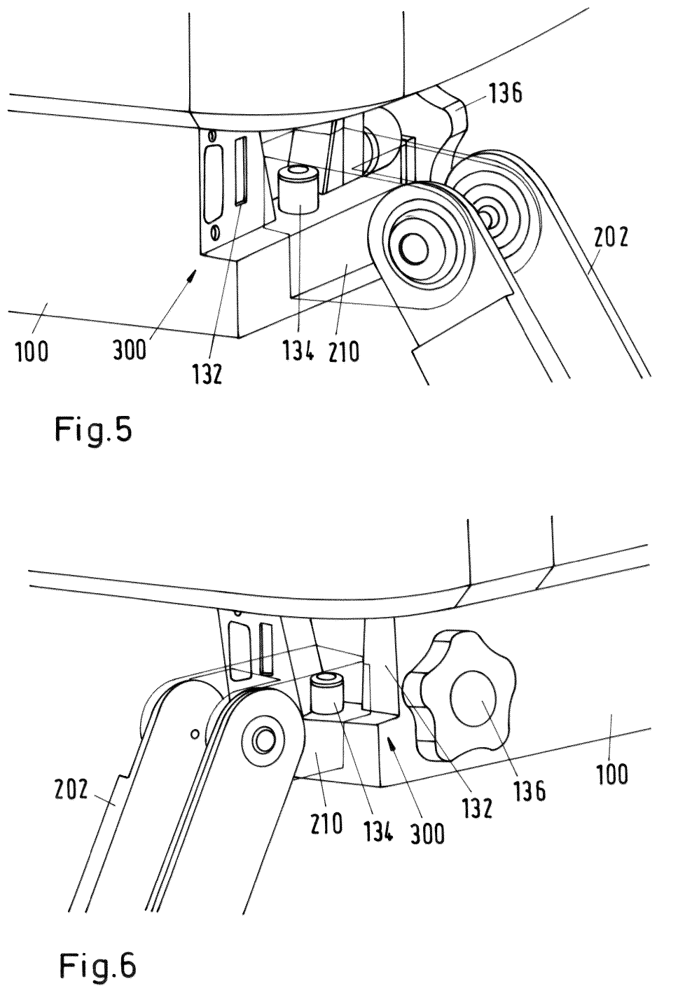

In the present case, the quick release interface 300 is in the form of a dovetail joint with a releasable latching device. This creates what is known as a V-lock. This can be identified in more detail in FIGS. 5 and 6.

In this case, the dovetail joint has a v-shaped taper, promoting the insertion of the second interface part 210 on the holding arm 202 into the first interface part 132 on the mount 100. The first interface part 132 of the mount 100 includes a latching member 134, which can mesh with a mating latching cutout (not shown in any more detail) on the second interface part 210 of the stereo image display device 200. The latching member 134 can be released from the latching cutout via a pushbutton or screwing, with the result that the quick release interface 300 is released, and the quick release interface 300 can be separated as a result. Further, the mating first interface part 132 of the mount 100 is assigned a securing device 136 for additionally securing the coupled quick release interface 300 in the present case. In the present case, this securing device 134 is formed by a securing screw which blocks an actuation of the latching device, in particular of the latching member 134.

The interface parts 210, 132 of the quick release interface 300 presently include electrical contacts and are configured to establish a data connection with the image recording device 104 and/or with a control computer coupled to the image recording device 104 when the quick release interface 300 is closed. This creates mechanical and electrical coupling of the stereo image display device 200 to the mount 100 or to the digital surgical microscope using the mount.

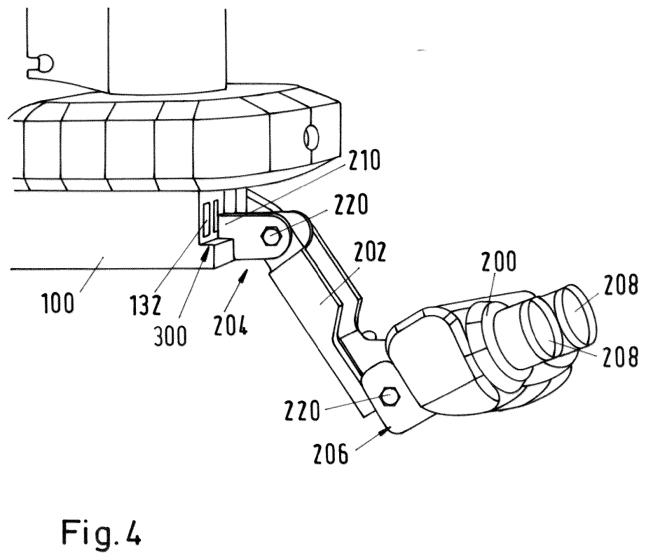

It is evident from FIGS. 2 to 4 that at least one joint 212 is present between the proximal end 204 and the distal end 206 of the holding arm 202 of the digital stereo image display device 200. Hence, the digital eyepieces 208 are each positively guided in a plane. Three joints 212, 220 are present in the configuration according to FIGS. 2 and 3. Two joints 220 are present in the variant according to FIG. 4. The joint 212 includes a joint axis 214 such that a distal partial arm 216 adjoining the distal end 206 is able to be pivoted in a common plane vis-à-vis a proximal partial arm 218 adjoining the proximal end 204. In the variant according to FIGS. 2 and 3, a respective further joint 220 is present at the proximal partial arm 218 and at the distal partial arm 216, the respective further joint allowing further pivoting in the plane. In the variant according to FIG. 4, the holding arm 202 also is of multi-part form and telescopic at the same time. A carriage which can slide within a part of the holding arm 202 is present to this end. An ergonomic work position for the surgeon can be realized by this configuration with the joints 220 or else with the telescopic device. The holding arms 202 of the shown digital stereo image display devices 200 are further distinguished in that they provide a channel for guiding electrical lines, the channel presently being realized in that the holding arm 202 has a u-shaped form in cross section; consequently, a u-shaped cable channel is present.

The chosen mechanism purposefully restricts degrees of freedom, and so the digital stereo image display device 200 can only be pivoted in one plane, whereby a common repositioning of the image recording device 104 and of the stereo image display device 200 can easily be undertaken.

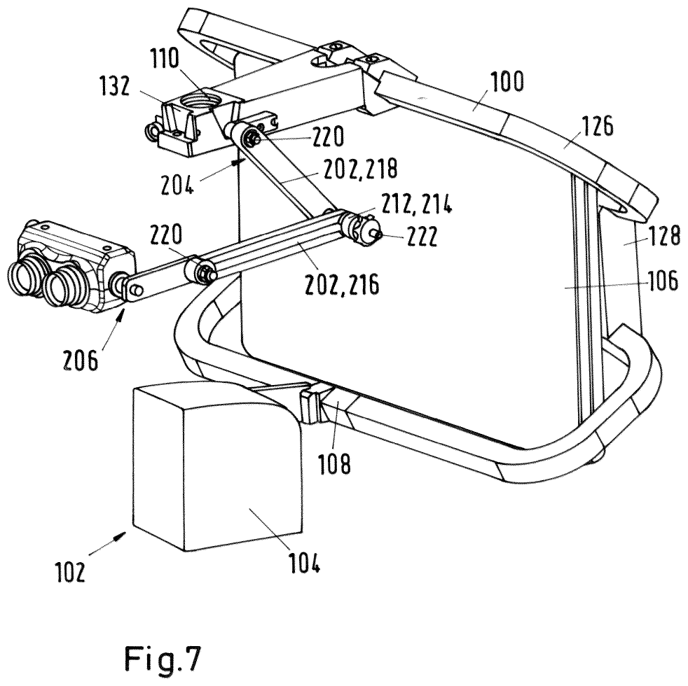

FIG. 7 shows an embodiment variant corresponding to that of FIG. 1, which merely differs in terms of the configuration of the holding arm 202 and its attachment on the digital eyepieces 208. Unlike the holding arm 202 according to FIGS. 1 to 6, the joint 212 in this case includes a joint axis 214 such that the distal partial arm 216 adjoining the distal end 206 is able to be pivoted in a first plane which is arranged parallel to a second plane in which the proximal partial arm 218 adjoining the proximal end 204 is able to be pivoted. An articulated chain extending between the proximal end 204 and the distal end 206 is realized in this way in the present case. Moreover, the holding arm 202 is presently attached to a side of the housing of the digital eyepieces 208. Thus, the attachment is off-center at the digital eyepieces 208.

To be able to lock and modify the pose of the digital eyepieces 208 in space again, the stereo image display device 200 according to FIG. 7 provides for the joint 212 and the further joints 220 to be assigned a releasable, for example screwable, locking means 222. In particular, these locking means 222 are also present in the variants of FIGS. 1 to 6.

Overall, the present disclosure is distinguished by improved ergonomics for a surgeon as the latter need not wear additional weight on their head, for example as would be the case with head-mounted displays (HMDs). The digital stereo image display device 200 can be assembled only when required and is consequently easily retrofittable. The embodiments according to the disclosure are easy to clean as a result of integrated cable routing. Additionally, a surgeon who is no longer able to recognize a three-dimensional image on the stereoscopic image display device 106, for example due to the effects of tiring, is then also easily able to assemble the digital stereo image display device 200 on the mount 100 and use the latter like a “conventional” surgical microscope.

It is understood that the foregoing description is that of the preferred embodiments of the invention and that various changes and modifications may be made thereto without departing from the spirit and scope of the invention as defined in the appended claims.

### LIST OF REFERENCE SIGNS

100 Mount
102 Optical system
104 Image recording device
106 Image display device
108 First attachment device (image recording device)
110 Second attachment device (attaching the mount)
124 First bracket
126 Second bracket
128 Connection piece
132 First interface part
134 Latching member
136 Securing device
200 Stereo image display device
202 Holding arm
204 Proximal end
206 Distal end
208 Digital eyepiece
210 Second interface part
212 Joint
214 Joint axis
216 Distal partial arm
218 Proximal partial arm
220 Further joint
222 Locking means
300 Quick release interface

1. A digital stereo image display device comprising:
a holding arm having a proximal end for attachment to a mount for an optical system with a digital image recording device;
said holding arm further having a distal end where two digital eyepieces are disposed and configured to display images captured by the digital image recording device from two different viewing angles; and,
an interface part of a quick release interface for releasable attachment to the mount being present at said proximal end.

2. The stereo image display device of claim 1, wherein said holding arm includes at least one joint between said proximal end and said distal end.

3. The stereo image display device of claim 2, wherein said two digital eyepieces are each positively guided in a plane.

4. The stereo image display device of claim 2, wherein said at least one joint defines a joint axis such that a distal partial arm adjoining said distal end is pivotable in a common plane vis-à-vis a proximal partial arm adjoining said proximal end.

5. The stereo image display device of claim 2, wherein said at least one joint defines a joint axis such that a distal partial arm adjoining said distal end is pivotable in a first plane arranged parallel to a second plane in which a proximal partial arm adjoining said proximal end is pivotable.

6. The stereo image display device of claim 4, wherein said holding arm includes a further joint disposed at least at one of said proximal partial arm and said distal partial arm, said at least one further joint allowing further pivoting of the two digital eyepieces in the plane.

7. The stereo image display device of claim 1, wherein said holding arm is multi-part and telescopic.

8. The stereo image display device of claim 2, wherein said at least one joint is assigned a releasable locking mechanism.

9. The stereo image display device of claim 6, wherein at least one of said at least one joint and said at least one further joint are assigned a releasable locking mechanism

10. The stereo image display device of claim 1, wherein the quick release interface forms an interlocking connection in a coupled state.

11. The stereo image display device of claim 10, wherein said quick release interface is a dovetail joint with a releasable latching device.

12. The stereo image display device of claim 1, wherein said interface part includes a plurality of electrical contacts and is configured to establish a data connection with at least one of the image recording device and a control computer coupled to the image recording device when said quick release interface is closed.

13. A system comprising:
a mount;
a digital stereo image display device having a holding arm having a proximal end for attachment to said mount for an optical system with a digital image recording device;
said holding arm further having a distal end where two digital eyepieces are disposed and configured to display images captured by the digital image recording device from two different viewing angles;
said digital stereo image display device further having a first interface part of a quick release interface for releasable attachment to said mount being disposed at said proximal end; and,
said mount having a second interface part of the quick release interface, wherein said second interface part is configured to mate with said first interface part of the digital stereo image display device.

14. The system of claim 13, wherein said second mating interface part of the mount is assigned a securing device for additionally securing the quick release interface when coupled.

15. A digital surgical microscope comprising:
a digital image recording device;
a system including a mount and a digital stereo image display device;
said digital stereo image display device having a holding arm having a proximal end for attachment to said mount for an optical system with said digital image recording device;
said holding arm further having a distal end where two digital eyepieces are disposed and configured to display images captured by the digital image recording device from two different viewing angles;
said digital stereo image display device further having a first interface part of a quick release interface for releasable attachment to said mount being disposed at said proximal end; and,
said mount having a second interface part of the quick release interface, wherein said second interface part is configured to mate with said first interface part of the digital stereo image display device.
